(19) United States
(12) Patent Application Publication
(10) Pub. No.: US 2009/0137881 A1
(43) Pub. Date: May 28, 2009

(54) Linked animal-human health visual analytics

(75) Inventors: David S. Ebert, West Lafayette, IN (US); Ross Larry Maciejewski, Lafayette, IN (US); Benjamin N. Tyner, Lafayette, IN (US); Yun Jang, West Lafayette, IN (US); William Cleveland, Lafayette, IN (US); Sandra Amass, Lafayette, IN (US)

Correspondence Address: Maginot, Moore & Beck;Chase Tower, Suite 3250, 111 Monument Circle, Indianapolis, IN, 46204, US

(73) Assignee: Purdue Research Foundation Office of Technologies Commercialization, West Lafayette, IN (US)

(21) Appl. No.: 12/286,756

(22) Filed: Oct. 1, 2008

Related U.S. Application Data
(63) Continuation of application No. 60/997,150, filed on Oct. 1, 2007.

(51) Int. Cl. A61B 5/00

(52) U.S. Cl. 600/300

(57) ABSTRACT

Coordinated animal-human health monitoring can provide an early warning system with fewer false alarms for naturally occurring disease outbreaks, as well as biological, chemical and environmental incidents. This monitoring requires the integration and analysis of multi-field, multi-scale and multi-source data sets. In order to better understand these data sets, models and measurements at different resolutions must be analyzed. To facilitate these investigations, we have created an application to provide a visual analytics framework for analyzing both human emergency room data and veterinary hospital data. Our integrated visual analytic tool links temporally varying geospatial visualization of animal and human patient health information with advanced statistical analysis of these multi-source data. Various statistical analysis techniques have been applied in conjunction with a spatio-temporal viewing window. Such an application provides researchers with the ability to visually search the data for clusters in both a statistical model view and a spatio-temporal view. Our interface provides a factor specification/filtering component to allow exploration of causal factors and spread patterns. In this paper, we will discuss the application of our linked animal-human visual analytics (LAHVA) tool to two specific case studies. The first case study is the effect of seasonal influenza and its correlation with different companion animals (e.g., cats, dogs) syndromes. Here we use data from the Indiana Network for Patient Care (INPC) and Banfield Pet Hospitals in an attempt to determine if there are correlations between respiratory syndromes representing the onset of seasonal influenza in humans and general respiratory syndromes in cats and dogs. Our second case study examines the effect of the release of industrial wastewater in a community through companion animal surveillance.

This application claims the benefit of U.S. Provisional Patent Application Ser. No. 60/997,150, filed Oct. 1, 2007, which is incorporated herein by reference.

### INTRODUCTION

The role of public health surveillance is to collect, analyze and interpret data about biological agents, diseases, risk factors and other health events in order to provide timely dissemination of collected information to decision makers. Surveillance activities share several common practices in the way data are collected, managed, transmitted, analyzed, accessed and disseminated. Surveillance methods that can detect disease at a pre-diagnostic stage are generally referenced to as syndromic because they have the ability to recognize outbreaks based on the symptoms and human behavior, sometimes prior to first contact with the healthcare system. As such, syndromic surveillance can be defined as the systematic and on-going collection, analysis and interpretation of data that precedes diagnosis.

In order to create better surveillance systems, it is important to know that an estimated 73% of emerging infectious diseases are zoonotic in origin [19, 24]. Thus, monitoring the companion animal population of a society (e.g. dogs, cats) can provide early warning signs for emerging diseases. In conjunction, exposures to many substances, such as pollutants, chemicals, allergens and natural toxins, originate from the environment and can have a detrimental effect on health. Companion animals are exposed to the same substances as humans and monitoring their health can function as a “canary in a coal mine” [25]. It has long been the goal of healthcare officials to identify and prevent hazardous exposures; however, lack of infrastructure and reportability in human health monitoring has hindered progress in this area. As such, we present a visual analytics environment that uses companion animal data in conjunction with human emergency room data as a detection system for emerging disease outbreaks and public health incidents.

Our application provides a framework for analyzing both human emergency room data and veterinary hospital data. Various statistical analysis techniques have been applied in conjunction with a spatio-temporal visualization system. Such an application provides researchers with the ability to visually search the data for clusters in both a statistical model view and a spatio-temporal view. By providing linked graphical and statistical analysis views for health care researchers and public health officials, we hope to improve event detection and response, while reducing false positives.

Our system uses emergency room data from the Indiana Network for Patient Care (INPC) and all general visits to the Banfield Pet Hospitals. The Indiana Network for Patient Care consists of five major hospital systems that serve more than 390,000 emergency room visits per year [I). The Banfield Pet Hospitals provide nationwide coverage with demographics distributed according to human population density. Coverage of Banfield Pet Hospitals is one location for every 5-mile radius containing 100,000 pet owners, and currently has greater than 600 veterinary hospitals located in 42 states that service approximately 70,000 pets per week. Hence, our system has nationwide syndromic coverage by using companion animals as sentinel surveillance, as well as a strong localized coverage in a major metropolitan area.

Currently, our work has focused on two case studies: 1) seasonal influenza and its correlation to general companion animal health, and 2) the effects of an industrial wastewater release on companion animals and the correlation to potential human health issues. In the case of seasonal influenza, early findings indicate that there may be a correlation between general dog respiratory symptoms and the onset of human influenza. In the case of the industrial wastewater release, several syndromes for both cats and dogs were analyzed and preliminary results indicated that the industrial wastewater release negatively influenced the health of companion animals in this region. Ongoing analysis is being performed in both cases before any definitive confirmations can be made.

Section 2 describes the motivation and necessity of improved syndromic surveillance while Section 3 discusses previous work in this area. Section 4 provides the details of the individual components of LAHVA. Section 5 outlines the details of the particular case studies we use to showcase our system, and Section 6 shows the application of our system to these case studies. Finally, we discuss conclusions and plans for future work in Section 7.

### BRIEF DESCRIPTION OF THE DRAWINGS

FIG. 1 shows a block diagram of an exemplary linked animal-human health visual analytics system in accordance with a first embodiment of the invention;

FIG. 2 shows exemplary viewing windows of the system of FIG. 1;

FIG. 3 shows a graph of trending of animal illnesses in the vicinity of a wastewater event;

FIG. 4 shows a graph of overlaid yearly seasonal components of correlations between dog respiratory syndromes and human respiratory syndromes;

FIG. 5 shows a plot of distance to a release point versus time, with horizontal bars indicating the 10% quantiles for each 21-day window.

FIG. 6a shows a graph of temporally varying window showing cases of human and companion animals showing signs of respiratory illnesses;

FIG. 6b shows a plot relating to the windows of FIG. 6a;

FIG. 7 shows another trending of animal illnesses identified as being potential indicators of adverse effects due to a wastewater release.

### MOTIVATION

Timely and accurate detection of unusual population health trends is a challenging problem requiring the analysis of data collected from disparate sources over time; These data sources vary widely in accuracy and reliability, and it is often the case that unusual health trends, such as outbreaks or poisonings, often have an incidence profile (signal) that is obscured by the statistical noise. For instance, the Indiana Public Health Emergency Surveillance System (PHESS) [9, 10] generates several daily potential outbreak alerts. However, only a handful of these alerts have proven to be significant events. Current systems, including those described in Section 3, are not capable of both high true positive rates (precision) and low false positive rates (recall).

In addition to suboptimal accuracy, current population monitoring systems face other challenges. Many existing systems do not leverage existing messaging and vocabulary standards such as Health Level 7 (HL7) and LOINC. Further, many systems require manual data input which further encumbers already overburdened public health and health care workers, and is infeasible as a long term solution. Other challenges include the lack of timely data acquisition, data quality concerns (e.g., duplicate records, typographical errors), and accurate data linkage.

Our system attempts to overcome many of these problems through the use of the Banfield Pet Hospital database. Banfield is a nationwide system with a geographical coverage similar to the human population. It captures veterinary visits in real-time for all Banfield practices, and this data can augment existing human syndromic surveillance efforts. Furthermore, we link to the Indiana Network for Patient Care (INPC) [I] database and monitor human health events in the Indianapolis metropolitan region.

### PREVIOUS WORK

Data from public health surveillance systems has long been recognized as providing meaningful measures for disease risks in populations [16, 21, 22]. In light of this, many systems have been developed to analyze this data and provide syndromic surveillance to epidemiologists. Some of the most popular of these systems are the Early Aberration Reporting System (EARS) [14], the Electronic Surveillance System for the Early Notification of Community-based Epidemics (ESSENCE) (17], and Biosense [18].

EARS was developed through the Centers for Disease Control and Prevention (CDC) and provides epidemiologists with several aberration detection methods. This system has been implemented in multiple state and local health departments throughout the United States and in several other countries. ESSENCE relies on both syndromic and nontraditional health information to provide early warnings of abnormal health conditions. This system is implemented in the national capital area, as well as many state health departments, and utilizes military and civilian healthcare information as the means of identifying abnormal outbreaks, Biosense is part of an national initiative to detect bio-terrorism. BioSense's main goal is to facilitate the sharing of automated detection and visualization algorithms through the creation of national standards. This implementation will include an internet-based software-system that includes both spatio-temporal and temporal analysis and currently operates in more than 20 cities.

These systems focus specifically on data collected on human health; however, this data is often encumbered by privacy concerns. Furthermore, many emergency rooms are not yet collecting electronic records, and those that do collect records often only do data analysis on the zip code level. In contrast, data collected at the Banfield Pet Hospitals is entered into a national database in real-time, allowing instant access for analysis. There are no privacy concerns for pets, so the exact location may be used for analysis instead of aggregation to the zip code level. As such our work focuses on syndromic surveillance by using companion animals as predictors to increase sensitivity and specificity.

The need for such companion animal monitoring has been outlined in presidential panels [7]; however, little work has been done in this area, Our system addresses this need by combining data from Banfield Pet Hospitals with INPC data. Unfortunately, though, not all methods used for syndromic surveillance in human data are appropriate for syndromic surveillance in companion animals. Due to the sparsity of pet visits with comparable syndromes, these data sources exhibit statistically different signal characteristics.

For human data, syndromic surveillance is done through means of aberration detection. Aberration detection is the change in the distribution or frequency of important health-related events when compared with historical data, and can be divided into two broad categories: case definition methods and pattern recognition methods. Case definition methods employ epidemiological experience to define syndromes of interest that would indicate an event. For pattern recognition methods, we employ the use of SatScan [15) which employs spatial, temporal, and spatio-temporal scan statistics to identify unusual disease clusters in a given population.

For aberration detection, most surveillance systems use long term data, three or more years, to calculate the expected historical value. However, historical data is not always available. As an approach for short term aberration detection, many systems employ the use of the CUSUM model (cumulative sum) [14, 13, 12]. CUSUM can be used for a short term (approximately 21 days) surveillance methods and due to the short length, seasonality factors are less important in the assessment of daily aberrations.

For companion animal data, we have tested several different aberration detection methods and report on both their benefits and shortcomings in the following sections.

### LINKED ANIMAL-HUMAN VISUAL ANALYTICS SYSTEM

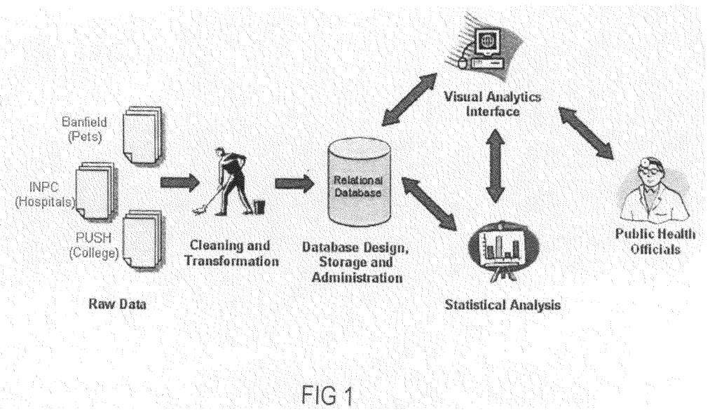

We have developed a system (LAHVA) that combines both human and animal health data for syndromic surveillance and aberration detection. Our system consists of three components: a date management component, a statistical analysis component and a visual analytics component as seen in FIG. 1. Our system directly accesses data from INPC and Banfield Pet Hospitals. The INPC data is updated daily in our database and the Banfield data is updated at regular intervals of 1-3 weeks. Currently, statistical models are pre-computed in R [20] and S-plus in order to evaluate their potential use. Future versions of the system will directly analyze the data through direct implementations of these methods.

### 4.1 Data Management

To support efficient and effective visualization analysis, we have built a data integration system that supports the transformation, management, and integration of raw human and animal health data. In the process, several data management issues were required: (1) cleaning and transformation of the data arriving from different data sources, (2) integration and correlation of data (e.g., hospitals and veterinary clinics), and (3) assurance that the data is used in a secure and privacy preserving way.

### 4.1.1 Data Preparation

Raw data arriving from emergency departments and Banfield Pet Hospitals is not directly usable. As such, several data preparation steps are applied, including data cleaning and transformation. Data cleaning is used for detecting and removing errors and inconsistencies from the raw data in order to improve the quality of data, and all data transformations are tracked and recorded. This preparation also allows us to provide feedback to our data providers in terms of how well their systems are being managed. Through this, several previously undetected data management issues have been resolved in their systems.

### 4.1.2 Data and Information Integration

Since the data comes from disparate sources stored in different formats, seamless and uniform querying and manipulating of this data is required. A critical challenge is matching and correlating the human and animal data coming from disparate sources using different naming conventions, relational schemas, and values that semantically may represent the same symptoms. While at this stage of the project, most of these issues are resolved in an ad hoc fashion, we are currently conducting research into different directions to solve these issues. Possible solutions include using the query logs from these different databases to automatically match their schemas.

### 4.1.3 Privacy-Preserving Data Sharing and Analysis

Once the data is processed and stored, data privacy and sharing concerns needed to be addressed. Since we are dealing with sensitive medical data, we may not make the assumption that access to this data can be granted without restrictions. In order to ensure that this data is protected from actions that violate the privacy of individuals, restrictions have been put into place. However, these restrictions need to also allow data extracted to be useful for our visual analytics system. We have to strike a balance between the need to preserve privacy and our capacity to enable rapid, accurate, comprehensible, and communicable analyses. Our current system uses traditional de-identification techniques to address this issue. We also are working on visual abstractions of the data where the information being visualized is transformed in such a way that it does not reveal any private information. This will complement our privacy preservation techniques applied at lower levels to the raw data.

### 4.2 Statistical Modeling

Once the data management system was created, it was necessary to address the statistical modeling problems of both human and companion animal data. As explained in Section 3, much work has been done on aberration detection in emergency room data. Unfortunately, many of these techniques are not easily applied to veterinary hospital data. In emergency room data, there are typically 9 to 11 chief complaints, most commonly consisting of: respiratory, gastro-intestinal, hemorrhagic, rash, fever, neurological, botulinic, shock/coma, and other. Multiple cases of these syndromes are present in emergency room data every day.

In contrast, the Banfield Pet Hospital data is more robust in that it contains detailed examination records of each pet that visited the hospital. These records may be searched for syndromes that are equivalent to emergency room chief complaints; however, the number of cases per day will often be zero. As such, common EARS analysis methods are not always applicable. In the following sections, we will discuss the statistical methods applied to the companion animal data and potential problems within.

### 4.2.1 Power Transformation

One method we applied to simplify our analysis was the application of a logarithm or power transformation to bring the data more in line with model assumptions [6]. In time series analysis, the logarithm transformation is widely applied when the mean is proportional to the standard deviation [3], and in cases where the data consists of counts following a Poisson distribution a square root transformation will approximately make the mean independent of the standard deviation. In each case, the transformations are necessary to simplify the modeling procedure.

Due to the zeros in the animal hospital data, a logarithm is not directly applicable. Naturally, log(x+1) was tried, but failed to eliminate the skewness on the right tail of the distribution for the number of observations. A square root transformation did not work either due to the skewness on the left tail caused by the zeros. Our experimental results suggest \sqrt{x+1} gives good performance in terms of stabilizing the variability and yielding a skew-free distribution in most cases.

### 4.2.2 Data Normalization

While a power transformation is useful for some analysis, others require data normalization to pull out the underlying trend. In the INPC and Pet Hospital data, daily counts are stored in our database and the daily counts can vary according to seasonal effects and increases in data collection capacity. Regular daily count plots tend to be very noisy and it is hard to identify abnormal characteristics. In order to analyze patterns of data over time, we apply a normalization to capture the aberrations in the data. To reduce the noisy patterns and to compensate for the different scaling in counts over time we typically use counts per week. For the denominator of our normalization, we use the sum of the daily counts for the past six months. This six month sliding window then allows us to observe the seasonal effects and larger trends while removing day of the week effects and smaller aberrations. As such, data normalization of this manner will not be applied when looking for short-term effects.

4.2.3 Aberration Detection for Sparse, Dependent Data For short term abberation detection, one statistical approach we applied was the use of CUSUM [14, 13, 12]. CUSUM is defined as the following.

$${S}_{t} = {max}_{( 0 , {S}_{t - 1} + \frac{{x}_{t} ( {μ}_{0} + k {σ}_{{x}_{1}} )}{{σ}_{{x}_{1}}} )} \qquad ( 1 )$$

where Sₜ is the current CUSUM, Sₜ₋₁ is the previous CUSUM, X_{I} is the count at the current time, μ₀ is the expected value, σ_{Xt} is the standard deviation, and k is the detectable shift from the mean. μ₀ and σ_{Xt} are computed according to the degree of sensitivity. We use three different models (C1, C2, C3) and each model uses different time period for the μ₀ and σ_{Xt} computations. For C1, the baseline period is Day₋₇, . . . , Day₋₁ and a flag is noted on Day₀. For C2, Day₋₉, . . . , Day₋₃ are used as the baseline and similarly, C3 uses Day₋₉, . . . , Day₋₃ as the baseline but an average of Day₋₂, . . . , Day₀ is used to detect the aberration. However, our Pet Hospital data has a relatively small number of counts and we use doubled baselines in order to avoid zero count for the baseline period. Here, we see the problems in analyzing our veterinary data using common human syndromic surveillance methods. The sparsity of the data requires a modification of the CUSUM, and may produce undesirable false positives.

As previously mentioned, zeros are common among daily counts of clinical signs among the Banfield pets within a given area (a radius of a few miles). Consequently, detection of aberrations must proceed over a large distance, or over longer time periods than a single day.

While it is common for this kind of data to exhibit both spatial and temporal variation, some variations may be uninteresting. For example, there may be temporal dynamics associated with a changing population that are not associated with a particular syndrome. To achieve reasonable sensitivity and specificity on important signals, it is necessary to first adequately model the unimportant effects. The problem is compounded by the fact that only local estimates of animal population are available.

Bootstrapping is a general-purpose robust alternative to parametric inference used when the analyst does not wish to make strong parametric assumptions about the data. In the words of its inventor [8], it “can by applied to complicated situations where parametric modeling and/or theoretical analysis is hopeless.” The idea is to sample the data with replacement in order to simulate the distribution of the data and functions thereof. When bootstrapping dependent data, care must be taken to preserve as much of the dependence structure as possible when doing the resampling. Typically this is done via a blocked approach; for a univariate time series the sampling units are then contiguous subseries drawn from the original data. Such a scheme is described by Carlstein et al. [4], with Hanna et al. [11] among the first applications.

Another statistical scheme to detect unusual variation in cases of symptomatic pets when operating in retrospective mode would be an application of quantile measures. For all pets within a radius of incidence, we identify all symptomatic encounters over a time window of t_{w} days after the alleged release at time t. Over the window [t, t+t_{w}), there is a distance to the epicenter associated with each symptomatic encounter, and our detection statistic S*ₜ is the radius inside which x % of the window's symptomatic cases occur. One imagines that an adverse event near the epicenter will cause the distribution of these distances to be shifted downward, and our approach seeks to detect such shifts over time.

Our reasons for using the quantile as a measure of location arc several-fold. First, it seems important to choose a statistic not dominated by animals far from the epicenter; a small quantile is likely to be more sensitive to aberrations close to the epicenter than would an arithmetic average, for example. Moreover, the distribution of the average distance is highly influenced by the choice of the radius, whereas the quantile should be less so. Of course, it is important not to choose a quantile so small that the bootstrap no longer applies; as an extreme case, the minimum is an example of a quantile whose distribution cannot be bootstrapped.

Though computationally intensive, the actual bootstrapping technique is rather straightforward: to obtain R null replicates of the statistic, one may resample R windows of length t_{w} days corresponding to null data and compute the statistic there, resulting in bootstrap replicates Sₜ⁽¹⁾, Sₜ⁽²⁾, . . . , Sₜ^{(r)}. In prospective mode, the null data occurs prior to the window under investigation; in retrospective mode, one may opt to include data from after the window as well. In any case the bootstrap significance associated with S* is then

pₜ=(1+number of {Sₜ⁽ⁱ⁾} exceeding Sₜ*)/(1+R) (2)

If the mild assumptions underlying the bootstrap hold, the nun distribution of pₜ is approximately discrete uniform over {1/R, 2/R, . . . , R/R}. Consequently, if there is no signal in the window under investigation, rejecting the null hypothesis when pₜ*≦α will result in a false alarm rate of α×100%. For prospective mode, one will need to update pt with the passage of time, and in this case a plot of pₜ* versus, is appropriate. In this case the {pₜ} are themselves correlated; moreover, the probability of at least one false alarm grows with, for fixed a. If the number of null windows is less than R (common for our analyses), then bootstrapping is unnecessary when only a p-value is required, since the bootstrap p-value will have expectation equal to the fraction of null windows with statistic at least as extreme as the observed value. However, statistics such as standard deviation can still benefit from the bootstrap in this situation.

The resampling of different null windows within the same radius assumes a stationary distribution across time. Of course this cannot be literally true due to effects such as a changing at-risk population; nevertheless, by not going too far back in time one may be able to minimize such temporal effects without needing to incorporate estimates of the population itself. If one is willing to assume the null distribution does not vary much with local geography, another strategy is to use a second epicenter as a control denominator, though this introduces another source of variability. For example, for a 20 mile radius, one may choose the second epicenter at least 40 miles away so that there is no overlap.

### 4.2.4 Seasonal-Trend Decomposition Based on Loess

The previous method discusses the identification of small signals; however, we are also interested in signal correlation. Our time series signals can be viewed as the sum of multiple trend components: a seasonal component and remainders. For each data signal, “trend components” are extracted to represent the long term trend and yearly seasonality using a seasonal decomposition of time series by loess (STL) [5]. Here, the “seasonal component” would represent the day-of-the-week effect.

Yᵢₜ=Tᵢₜ+Sᵢₜ+Dᵢₜ+rᵢₜ (3)

where for the ith series, Yᵢₜ is the original series, Tᵢₜ is the long term trend, Sᵢₜ is the yearly seasonality, Dᵢₜ, is the day-of-the-week effect, and rᵢₜ, is the remainder. We can then look at the correlation between the extracted components to see if they have any potential effects on each other.

### 4.3 Visual Analytics

Our visual analytics system, LAHVA, takes advantage of both the data-management and statistical modeling components presented above. An initial direct access query to the database is done, and human hospitals, veterinary hospitals and individual animal locations are displayed on an interactive map. Statistical plots are pre-computed and linked to the factor specification and filtering components in the system.

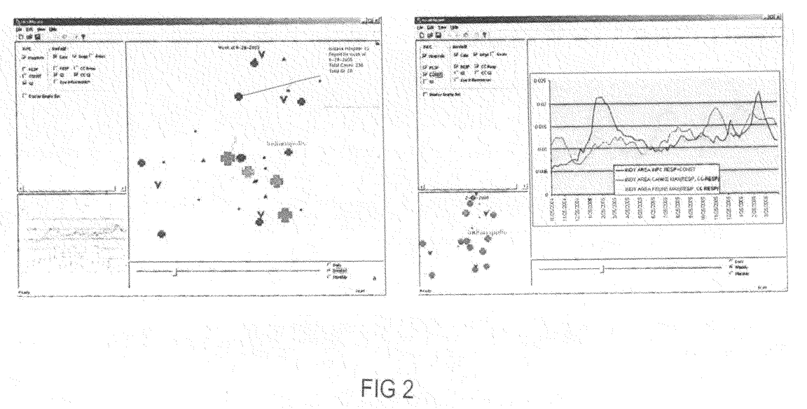

In FIG. 2, we see the typical LAHVA viewing windows. Emergency rooms are represented by crosses, veterinary hospitals are represented by the large V's, cats are triangles, and dogs are circles. For the emergency rooms and veterinary hospitals, the size and color are determined by the number of cases seen on that given time period, normalized by either the six-month sliding window previously discussed, or modified by a power transformation. As more cases of a particular syndrome are encountered on the specified time period, the colors change from green to red and the glyph area increases proportionally to the number of cases. Glyph scaling in the images is also enlarged to help preserve privacy and the scaling during use can be set smaller for higher specificity or larger to help signal alerts. The time period can be specified as daily, weekly or monthly using the controls on the bottom right near the slider, and the slider allows users to move forward and backwards in time.

The case selection and factors are determined by the check boxes in the upper left corner and marc factors are in the process of being added. Further information can be obtained by left-clicking on a human or animal hospital glyph. This opens an information screen that details the patient records for the specified time period, see FIG. 2 (Left).

For the cats and dogs, red represents respiratory syndromes, blue would represent gastro-intestinal syndromes and green would represent eye-inflammation syndromes. For prototyping purposes, the lower left window contains pre-computed plots of the data for varying factors. The main window contains the time-varying geo-spatial interface. Time is controlled by the slider on the lower portion of the window. By clicking on the statistical window plot, the main window and lower-left window of the system will switch allowing for different types of analysis as seen in FIG. 2 (Right). Future versions of this system will include more robust mapping features and interactive statistical analysis components.

### CASE STUDIES

In order to evaluate our system and test different aberration detection methods, two case studies were chosen. The first case study uses both the human and companion animal data for enhanced syndromic surveillance, while the second case uses only the companion animal data to demonstrate the benefits of this population in syndromic surveillance.

### 5.1 The Effects of Seasonal Influenza

Our first case study focuses on correlations between companion animal and human illnesses. Particularly, we analyze seasonal influenza through emergency room department chief complaints. Much work has been already been done on identifying seasonal influenza via chief complaint (e.g., [23, 2]). However, little has been done in comparing equivalent flu-like syndromes in companion animals. For our work, we arc using eighteen emergency rooms based in the Indianapolis metropolitan region. Trends of cat and dog illnesses in Indiana and bordering metropolitan areas were analyzed. For comparison, we focused on cats and dogs reporting respiratory syndromes and compare how these would correlate to emergency room chief complaints of respiratory syndromes.

### 5.2 Assessing Effects of a Chemical Release

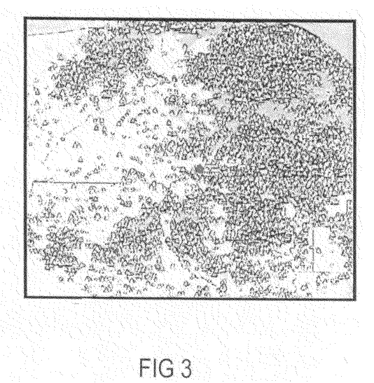

Our second case study focuses on using pets as sentinels to detect unusual events. Here, we focus on the release of industrial wastewater. The site in question has been anonymized and is shown in FIG. 3. The release center is denoted as a red diamond.

In order to examine the effects of this release, the local Department of Health led an investigation in the region. This region has a human population of approximately 8,500; and the combined human population of the nearby communities is approximately 28,000. Unfortunately, lack of human health data sources led the local Department of Health to assess these effects through a self-reported survey. In contrast, our study focuses on pets in a twenty-mile radius surrounding the site using data from Banfield, the pet hospital. We have a population of 74,660 dogs and 21,202 cats in this area as well as patient records prior to and following the release dates. Distributions of this population can be seen in FIG. 3.

### RESULTS

In order to test the functionality of our system, LAHVA was applied to the case studies described in Section 5. Various statistical methods were used to test their functionality in conjunction with the geospatial temporal viewing window.

### 6.1 Seasonal Influenza Analysis

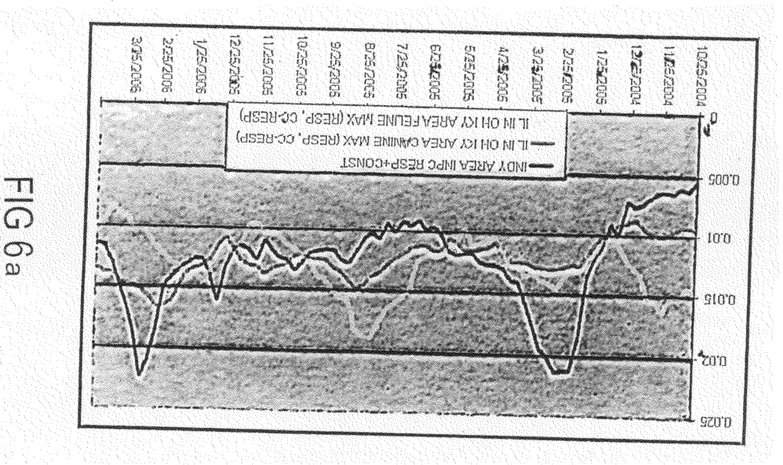

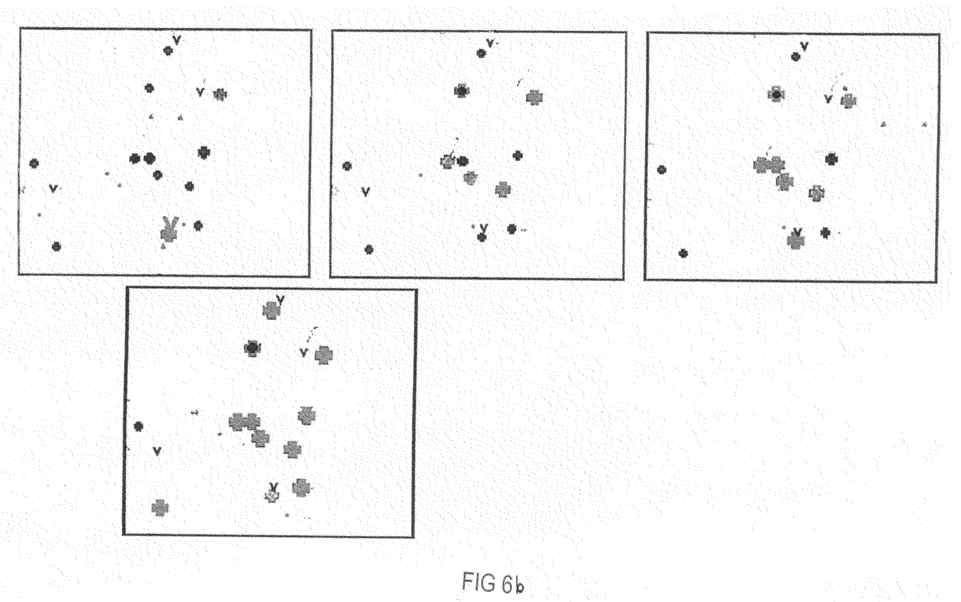

Our first case study was an analysis of seasonal influenza using LAHVA. In FIG. 6a we show the temporally varying window centered over the Indianapolis metropolitan area. The factor specification is showing cases of human and companion animals showing signs of respiratory illnesses. From LAHVA, one can easily identify the onset of seasonal influenza as the hospitals begin showing signs of increased respiratory cases. Viewing the statistical plot of FIG. 6b coupled with this allows us to see the overlying trend of respiratory syndromes in this area over a multi-year period. The blue line in the plot represents the INPC hospitals, the magenta line represents dogs with respiratory syndromes and the yellow represents cats with respiratory syndromes.

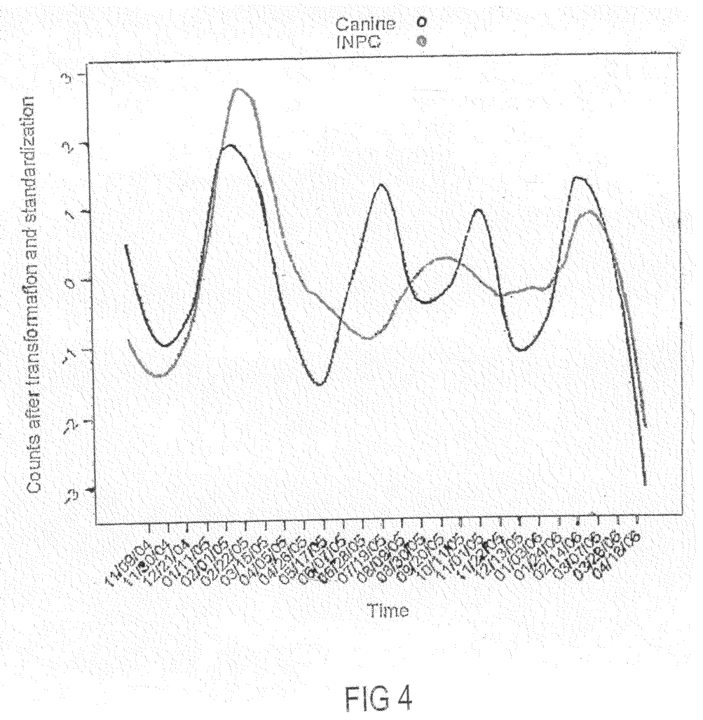

We also applied the STL analysis to see if there were correlations between dog respiratory syndromes and human respiratory syndromes. The yearly seasonal components for these two series are overlaid in FIG. 4. Here, we can see the similarity between the two. The data are standardized by subtracting the mean and dividing by standard deviation for visualization and comparison purposes. The grey bars are used to roughly illustrate the local maximum values over time providing evidence that respiratory symptoms in dogs occur approximately 10 days earlier than that of the humans in regular years.

### 6.2 Industrial Wastewater Release Analysis

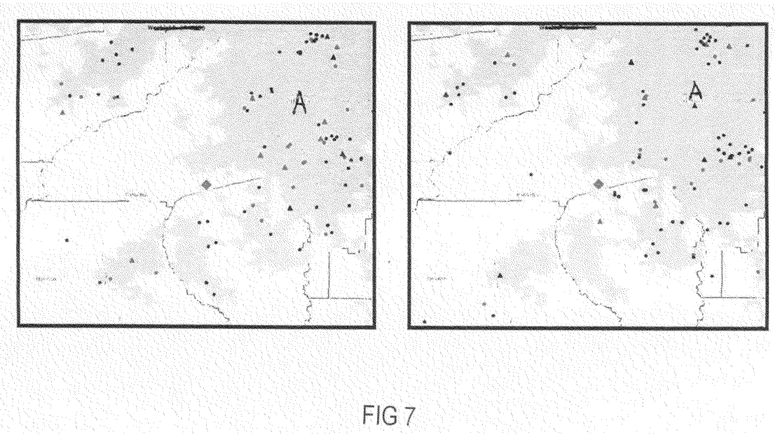

Our second case study analyzes the effects of an industrial wastewater release through companion animal surveillance. Three syndromes were identified as being potential indicators of adverse effects due to a release: eye inflammation, respiratory, and gastrointestinal. In FIG. 7 we see an area within a 20 mile radius of the spill. Cats are triangles and dogs are circles. In the week following the spill, what seems to be an unusual amount of eye-inflammation cases appear near the source. FIG. 7 (Left) is one week prior to the spill (June 22-28). FIG. 7 (Right) is the week starting the day of the spill (June 29-July 5). The green glyphs represent animals with eye-inflammation.

Once a problem is visually identified in our system, different statistical analyses can be run to confirm or deny problems in that area. CUSUM was applied to the data to determine if any alerts would be generated for eye-inflammation in this area. FIG. 7 of Appendix A in U.S. Provisional Patent Application Ser. No. 60/997,150, filed Oct. 1, 2007 shows the resultant CUSUM plots using CUSUM2. Due to the small number of eye-inflammation cases seen over the course of a year it is difficult to determine any direct information from applying CUSUM directly to the pet syndrome data. Current work is being done to find ways to potentially better apply CUSUM to the data.

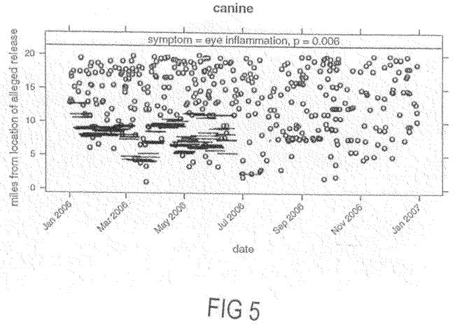

Due to the data sparsity, the application of CUSUM was not effective in this case. In order to further verify that problems with eye-inflammation occurred, the bootstrapping method discussed in Section 5.2.3 was applied. To illustrate the procedure and effect size FIG. 5 shows a plot of distance to the alleged release point versus time, with horizontal bars indicating the 10% quantiles for each 21-day window. This results are shown in Table 1, and indicate that eye-inflammation in dogs was significant near the release in our time period of interest.

TABLE 1
Summary of the bootstrap analysis findings

| species | statistic | eye inflammation |
|---|---|---|
| canine | mean 10% quantile before | 8.035 |
| | 10% quantile during | 2.365 |
| | 1-sided bootstrap p-value | 0.006 |
| feline | mean 10% quantile before | 11.195 |
| | 10% quantile during | 17.531 |
| | 1-sided bootstrap p-value | 0.909 |

### CONCLUSIONS AND FUTURE WORK

Our work has demonstrated the benefits of creating a linked visual statistical analysis system for health surveillance, and our methodologies are currently being applied to other case studies. It is clear that using companion animals for syndromic surveillance has great potential for early aberration detection; however, more work is needed to determine appropriate methodologies for using companion animals as sentinels. Our system has demonstrated the use of applied visual analytics through two different case studies. In both cases, the visuals allow users to easily locate potential problems in a region and then apply further statistical analyses to confirm their suspicions.

In the case of the effects of human influenza on general dog respiratory symptoms, we were able to find early signs indicating that there may be correlations between these events. In the case of the industrial wastewater spill, we were able do identify problem areas. From these problem areas, statistical tests were generated and we were able to verify what was seen visually.

While our current work has been retrospective, we intend to modify the system and integrate our statistical models for better interactivity. By doing this, we can provide health care officials and epidemiologists with tools to monitor varying regions of the country and provide better detection for potential disease outbreaks and health incidents.

Future work will focus on verification of these case study results, as well as others, and system enhancements to LAHVA. Current plans include adding the statistical analysis features directly to LAHVA and allowing users to interactively select areas of the map to analyze for potential health issues. Also, given the discreteness of illness data, i.e., records only exist on the day pets visit, we also plan to add time ghosting for an approximated contagious period. This period will be based on syndrome and interactively modifiable.

### REFERENCES

[I] p, G. Biondich and S. I. Grannis. The Indiana network for patient care: An integrated clinical information system informed by over thirty years of experience. Public Health Management Practices, pages 81-86, November 2004,
[2] F. Bourgeois, K. Olson, J. Brownsten, A. McAdam, and K. Mandl. Validation of syndromic surveillance for respiratory infections. Annals of Emergency Medicine, 47:265-271, 2006.
[3] P. J. Brockwell and R. A. Davis. Introduction to Time Series and Forecasting (2nd edition), Springer, 2003.
[4] E. Carlstein, The use of subseries values for estimating the variance: of a general statistic from a stationary sequence. Annals of Statistics, 14:1171-1179, 1986.
[5] R. B. Cleveland, W. S, Cleveland, J, McRae, and I. Terpenning. Stl: A seasonal-trend decomposition procedure based on loess. Journal of Official Statistics, 6:3-73, 1990.
[6] W. S. Cleveland. Visualizing Data. Hobart Press, 1993.
[7] N. R. Council. Animals as Sentinels of Environmental Health Hazards. National Academy Press, Washington, D.C., 1991. Library of Congress Catalog No. 91-61734.
[8] B. Efron. The Jackknife, the Bootstrap and Other Resampling Plans. SIAM, Philadelphia, 1982.
[9] S. J. Grannis, P. G. Biondich, B, W. Mamlin, G. Wilson, L. Jones, and J. M. Overhage. How disease surveillance systems can serve as practical building blocks for a health information infrastructure: the Indiana experience. In AMIA Annual Symposium, pages 286-290, 2005.
[10] S, J. Grannis, M. Wade, J. Gibson, and J. M. Overhage. The Indiana public health emergency surveillance system: Ongoing progress, early findings, and future directions. In American Medical Informatics Association, 2006.
[11] S. R. Hanna. Confidence limits for air quality model evaluations, as estimated by bootstrap and jackknife resampling methods. Atmospheric Environment, 23:1385-1398, 1989.
[12] L. Hutwagner, T. Browne, G. M. Seeman, and A. T, Fleischauer. Comparing aberration detection methods with simulated data. Emerging Infectious Diseases, 11(2):314-316, February 2005.
[13] L. C. Hutwagner, W. W. Thompsom, G. M. Seeman, and T, Treadwell. A simulation model for assessing aberration detection methods used in public health surveillance for systems with limited baselines, Statistics in Medicine, 24(4):543-550, February 2005.
[14] L. C. Hutwagner, W. W. Thompson, and G. M. Seeman. The bioterrorism preparedness and response early aberration reporting system (ears). Journal of Urban Health, 80(2):i89-i96, 2001.
[15] M. Kulldorff. A spatial scan statistic. Communications in Statistics: Theory and Methods, 26, 1997.
[16] A. D. Langmuir. The surveillance of communicable diseases of national importance. New England. Journal of Medicine, 268: 182-192, 1963.
[17] J. S. Lombardo. A systems overview of the electronic surveillance system for the early notification of community based epidemics (ESSENCE II). Journal of Urban Health, 80:32-42, 2003.
[18] J. W. Loonsk. Biosense—a national initiative for early detection and quantification of public health emergencies. MMWR, 53:53-55, 2004.
[19]°M. Pappaioanou, T. Gomez, and C. Drenzek. New and emerging zoonoses. Emerging infectious Diseases, 10(11), November 2004.
[20] R Development Core Team. R: A Language and Environment for Statistical Computing. R Foundation for Statistical Computing, Vienna, Austria, 2007. ISBN 3-900051-07-0.
[21] S. B. Thacker and R. L. Berkelman. Public health surveillance in the united states. Epidemiology Review, 10:164-190, 1988.
[22] S. B. Thacker, R. L. Berkelman, and D. F. Stroup. The science of public health surveillance. Journal of Public Health Policy, 10:187203, 1989.
[23] F.-C. Tsui, M. M. Wagner, V. Dato, and C.-C. H. Chang. Value of ICD-9-Coded Chief Complaints for Detection of Epidemics. J Am Med Inform Assoc, 9(90061):S41-47, 2002.
[24] M. E. J. Woolhouse and S. Gowtage-Sequeria. Host range and emerging and reemerging pathogens. Emerging Infectious Diseases, 11(0997), November 2005.
[25] R. D, Zane. Syndromic surveillance: A canary in the coal mine? Journal Watch Emergency Medicine, pages 265-271, April 2006. Further detail is provided by way of an exemplary case study, which is set in U.S. Provisional Patent Application Ser. No. 60/997,150, filed Oct. 1, 2007, which is incorporated in its entirety herein by reference.

We claim:

1. A method comprising:
a) obtaining data regarding patient data for a first species and patient data for a second species, the first species patient data comprising reported symptom incidences for a first species, and the second species patient data comprising reported symptom incidences for a second species;
b) performing statistical analysis on the first species patient data and the second species patient data to obtain refined first species patient data and refined second species patient data;
c) causing a display of a visual graphic including a map background and a plurality of color-coded symbols, each symbol representative of a select one of either species or symptom, and each color representative of the other species or symptom, the location of the color-coded symbols having a screen location relative to the map background corresponding to patient location information.

2. The method of claim 1, further comprising d) receiving input identifying a time period, and wherein step c) further comprises cause the display of color-coded symbols such that the color-coded symbols correspond to first species patient data and second species patient data for symptom incidences within the identified time period.

3. The method of claim 1, wherein step d) further comprises displaying an interactive slide control, and wherein a duration of the time period is determined by a user-selected position of the interactive slide control.

4. The method of claim 1, wherein step b) further comprises performing a power transformation on the first species patient data.

5. The method of claim 1, wherein step b) further comprises performing data normalization on the first species patient data.

6. The method of claim 5, wherein step b) further comprises employing a cumulative sum model on the first species patient data.

7. The method of claim 5, wherein step b) further comprises employing quantile measures on the first species patient data.
